(19) United States
(12) Patent Application Publication
(10) Pub. No.: US 2025/0381003 A1
(43) Pub. Date: Dec. 18, 2025

(54) EXTREMITY REHABILITATION METHOD AND ROBOTIC DEVICE USING THE SAME

(71) Applicant: Futronics (NA) Corporation, Pasadena, CA (US)

(72) Inventors: Wenda Xu, Pasadena, CA (US); Yunfei Guo, Pasadena, CA (US); Fulan Li, Pasadena, CA (US); Baiyu Wang, Pasadena, CA (US); Fangyun Zhao, Pasadena, CA (US); Chengkun Zhang, Pasadena, CA (US)

(21) Appl. No.: 18/743,106

(22) Filed: Jun. 14, 2024

(51) Int. Cl. A61B 34/00; A61B 34/30

(52) U.S. Cl. CPC A61B 34/77; A61B 2034/305

(57) ABSTRACT

Robotic device control for assisting a user in performing extremity rehabilitation is disclosed. A method controls a robotic device to assist extremity rehabilitation by: detecting, through a force sensor of a handle part of the robotic device that is coupled to an end effector of the robotic device for moving the end effector, an external force; decomposing the detected external force into a tangential force and a radial force; scaling, according to a distance between a current position of the handle part and a desired trajectory of the handle part, the radial force; calculating, based on a sum of the tangential force and the scaled radial force, a motor velocity for motors of the end effector; and providing a velocity instruction based on the calculated motor velocity for rotating the motors to move the end effector.

### TECHNICAL FIELD

The present disclosure relates to robotic technology, and particularly to an extremity rehabilitation method and a robotic device using the same.

### BACKGROUND

In the realm of upper extremity rehabilitation, the integration of technology has paved the way for innovative approaches to enhance patient outcomes. One such advancement involves the utilization of endpoint-based extremity rehabilitation devices capable of providing customized force assistance or resistance along predefined rehabilitation trajectories. Central to the efficacy of these devices is the design and implementation of adaptive force controls that dynamically adjust force parameters based on real-time feedback received through sensors.

Most extremity rehabilitation devices with end effector on the market either achieved fully powered trajectory following—meaning the device moves automatically and perfectly on the planned trajectory and users have no autonomy, fully free-form mode—meaning the users can operate the device without any constraints, or tiered resistance mode—meaning the users are offered with different levels of resistance level so users need to apply strength. However, most adaptive control mechanism of these devices requires operating by integrating additional sensors like electromyography (EMG) sensors which increases its complexity.

### BRIEF DESCRIPTION OF DRAWINGS

In order to more clearly illustrate the technical solutions in this embodiment, the drawings used in the embodiments or the description of the prior art will be briefly introduced below. In the drawing(s), like reference numerals designate corresponding parts throughout the figures. It should be understood that, the drawings in the following description are only examples of the present disclosure. For those skilled in the art, other drawings can be obtained based on these drawings without creative works.

FIG. 1 is a schematic diagram of a scenario of extremity rehabilitation using a robotic device and a display device according to some embodiments of the present disclosure.

FIG. 2 is a schematic diagram of the robotic device of FIG. 1.

FIG. 3 is a schematic block diagram illustrating the robotic device of FIG. 2.

FIG. 4 is a schematic block diagram of an example of performing extremity rehabilitation using the robotic device of FIG. 3.

FIG. 5 is a flow chart of an example of obtaining and decomposing external force according to some embodiments of the present disclosure.

FIG. 6 is a schematic diagram of a screen for selecting rehabilitation trajectory according to some embodiments of the present disclosure.

FIG. 7 is a schematic diagram of decomposing external force according to some embodiments of the present disclosure.

FIG. 8 is a flow chart of an example of providing customized force according to some embodiments of the present disclosure.

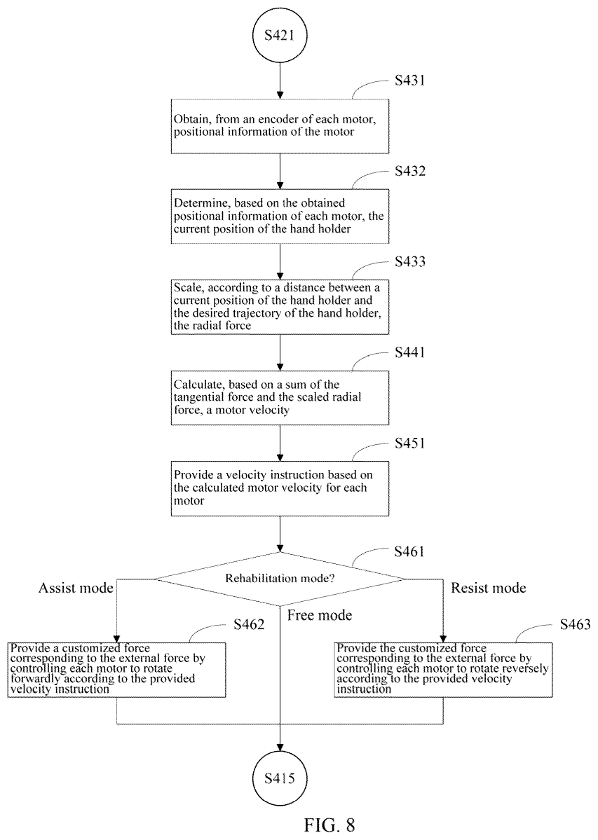

### DETAILED DESCRIPTION

In order to make the objects, features and advantages of the present disclosure more obvious and easy to understand, the technical solutions in this embodiment will be clearly and completely described below with reference to the drawings. Apparently, the described embodiments are part of the embodiments of the present disclosure, not all of the embodiments. All other embodiments obtained by those skilled in the art based on the embodiments of the present disclosure without creative efforts are within the scope of the present disclosure.

It is to be understood that, when used in the description and the appended claims of the present disclosure, the terms “including”, “comprising”, “having” and their variations indicate the presence of stated features, integers, steps, operations, elements and/or components, but do not preclude the presence or addition of one or a plurality of other features, integers, steps, operations, elements, components and/or combinations thereof.

It is also to be understood that, the terminology used in the description of the present disclosure is only for the purpose of describing particular embodiments and is not intended to limit the present disclosure. As used in the description and the appended claims of the present disclosure, the singular forms “one”, “a”, and “the” are intended to include the plural forms as well, unless the context clearly indicates otherwise.

It is also to be further understood that the term “and/or” used in the description and the appended claims of the present disclosure refers to any combination of one or more of the associated listed items and all possible combinations, and includes such combinations.

In the present disclosure, the terms “first”, “second”, and “third” are for descriptive purposes only, and are not to be comprehended as indicating or implying the relative importance or implicitly indicating the amount of technical features indicated. Thus, the feature limited by “first”, “second”, and “third” may include at least one of the feature either explicitly or implicitly. In the description of the present disclosure, the meaning of “a plurality” is at least two, for example, two, three, and the like, unless specifically defined otherwise.

In the present disclosure, the descriptions of “one embodiment”, “some embodiments” or the like described in the specification mean that one or more embodiments of the present disclosure can include particular features, structures, or characteristics which are related to the descriptions of the descripted embodiments. Therefore, the sentences “in one embodiment”, “in some embodiments”, “in other embodiments”, “in other embodiments” and the like that appear in different places of the specification do not mean that descripted embodiments should be referred by all other embodiments, but instead be referred by “one or more but not all other embodiments” unless otherwise specifically emphasized.

The present disclosure relates to controlling a robotic device for assisting a user in performing extremity rehabilitation. As used herein, the term “robotic device” refers to a machine such as an extremity rehabilitation device that includes mechanical components, logic circuitry, computing components, software and/or other specialized components that desired and/or measured force, torque, position, orientation, velocity, and/or angular velocity information is processed by a computing source and such computing source is used to control the force, torque, position, orientation, velocity, angular velocity, and/or physical configuration of the device. The term “end effector” refers to a part of a robotic device interacting with its environment to perform its functions. The term “sensor” refers to a device, module, machine, or subsystem such as force sensor (e.g., torque sensor) and image sensor (e.g., camera) whose purpose is to detect events or changes in its environment and send the information to other electronics (e.g., processor).

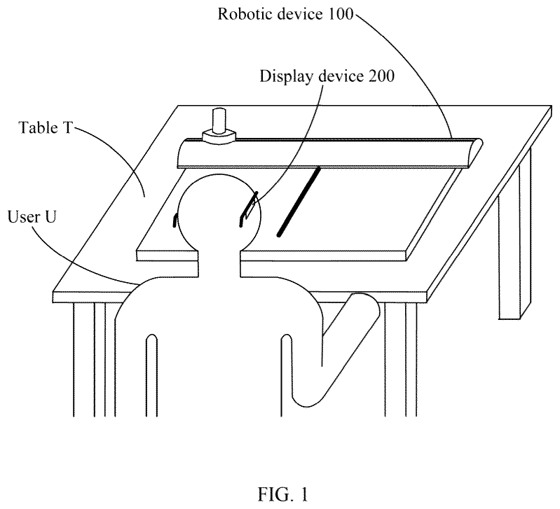

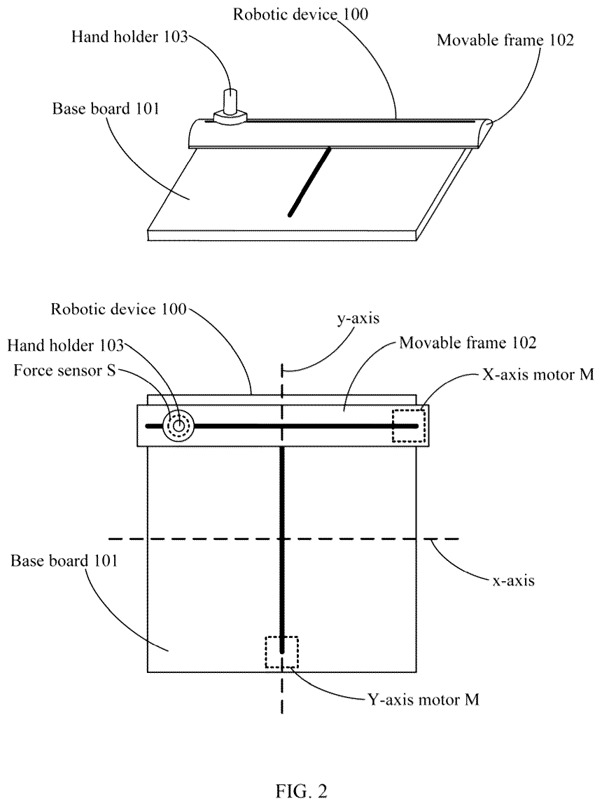

FIG. 1 is a schematic diagram of a scenario of extremity rehabilitation using a robotic device 100 and a display device 200 according to some embodiments of the present. In the scenario (e.g., nursing home, hospital, and home) for a user U (e.g., a patient needs rehabilitation because of an illness such as a stroke) to perform rehab activities, rehabilitation equipment like the robotic device 100 (and auxiliary equipment like the display device 200) may be provided to realize physical therapies. FIG. 2 is a schematic diagram of the robotic device 100 of FIG. 1. In some embodiments, the robotic device 100 is an extremity rehabilitation device that can provide extremity rehabilitation-related functions by putting on a table T or other suitable supporter for the user U to operate in a suitable posture like sitting to perform upper limb rehabilitation activities, which may include a base board 101, a movable frame 102 mounted on the base board 101 in a movable manner such as slidable in a y-axis direction (i.e., the direction aligned with a y-axis of a coordinate system of the robotic device 100) along a rail of the base board 101, and a hand holder 103 (i.e., a handle) mounted on the movable frame 102 in a movable manner such as slidable in an x-axis direction (i.e., the direction aligned with an x-axis of the coordinate system of the robotic device 100) along a rail of the movable frame 102. The base board 101 and the movable frame 102 jointly form an end effector of the robotic device 100. The base board 101 includes a y-axis motor M for being rotated to move the movable frame 102 along the y-direction, and the movable frame 102 includes an x-axis motor M for being rotated to move the hand holder 103 along the x-direction. The hand holder 103 is for supporting an upper limb of the user U, which may include a force sensor S. When the user U operates the robotic device 100 through the hand holder 103, the upper limb is moved upon the base board 101 with the movement of the hand holder 103, thereby performing the upper limb rehabilitation activities.

The robotic device 100 detects forces from the user U through the force sensor S so as to provide force feedbacks corresponding to the detected forces to simulate real-world physical touch by way of, for example, motorized motion or resistance. The display device 200 is a headset that facilitates the user U to perform the upper limb rehabilitation activities by, for example, providing related textual/audio/graphical instructions, introductions, suggestions, or displaying related virtual reality (VR)/augmented reality (AR) images or the like, which may include a stereoscopic display to provide separate images for each eye of the user U, a stereo, and sensor(s) like a camera for capturing images of the surroundings of the user U, or an accelerometer and a gyroscope for tracking the pose of the head of the user U to match the orientation of a virtual camera with the positions of the eyes of the user U in the real world so as to simulate the physical presence of the user U in a virtual environment so that the user U is able to look around the artificial world, move around in it, and interact with virtual features or items. The display device 200 may provide different display modes such as a real mode, an AR mode, and a VR mode for the user U to switch through, for example, physical button(s) or remote control of the display device 200. In other embodiments, the display device 200 may be other display device like a display screen or a multi-projected environment to generate realistic images. In addition, other feedbacks like haptic feedback may be provided through the robotic device 100 or other devices like joysticks.

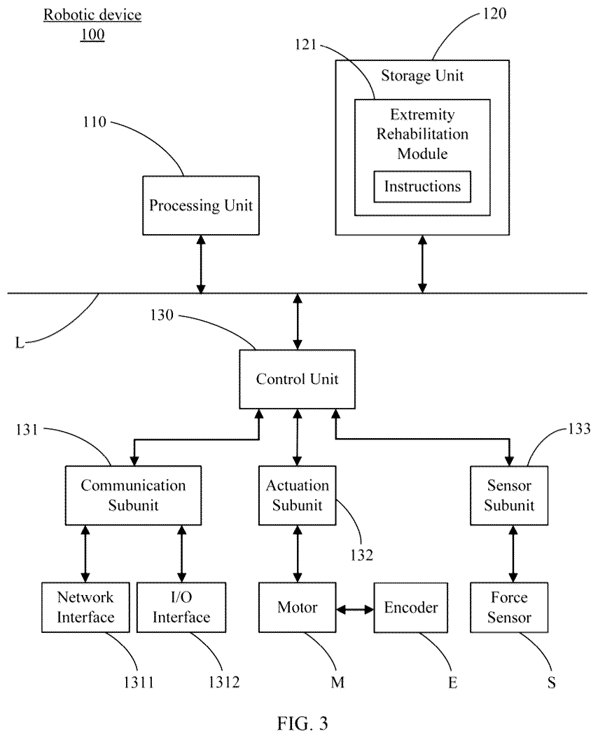

FIG. 3 is a schematic block diagram illustrating the robotic device 100 of FIG. 2. The robotic device 100 may include a processing unit 110, a storage unit 120, and a control unit 110 that communicate over one or more communication buses or signal lines L. It should be noted that, the robotic device 100 is only one example of robotic device, and the robotic device 100 may have more or fewer components (e.g., unit, subunits, and modules) than shown in above or below, may combine two or more components, or may have a different configuration or arrangement of the components. The processing unit 110 executes various (sets of) instructions stored in the storage unit 120 that may be in form of software programs to perform various functions for the robotic device 100 and to process related data, which may include one or more processors (e.g., CPU). The storage unit 120 may include one or more memories (e.g., high-speed random access memory (RAM) and non-transitory memory), one or more memory controllers, and one or more non-transitory computer readable storage mediums (e.g., solid-state drive (SSD) or hard disk drive). The control unit 130 may include various controllers (e.g., network interface controller, display controller, and physical button controller) and peripherals interface for coupling the input and output peripheral of the robotic device 100, for example, external port (e.g., USB), wireless communication circuit (e.g., RF communication circuit), audio circuit (e.g., speaker circuit), sensor (e.g., inertial measurement unit (IMU)), and the like, to the processing unit 110 and the storage unit 120. In some embodiments, the storage unit 120 may include an extremity rehabilitation module 121 for implementing upper limb rehabilitation functions related to the above-mentioned upper limb rehabilitation activities (e.g., mechanical/electronical functions to control the robotic device 100 so as to enable the user U to perform the upper limb rehabilitation activities), which may be stored in the one or more memories (and the one or more non-transitory computer readable storage mediums. The extremity rehabilitation module 121 may be a software module (of the operation system of the robotic device 100), which has instructions (e.g., instruction for actuating the motors M of the robotic device 100) for implementing the above-mentioned upper limb rehabilitation functions.

The robotic device 100 may further include a communication subunit 131 and an actuation subunit 132. The communication subunit 131 and the actuation subunit 132 communicate with the control unit 110 over one or more communication buses or signal lines that may be the same or at least partially different from the above-mentioned one or more communication buses or signal lines L. The communication subunit 131 is coupled to communication interfaces of the robotic device 100, for example, network interface(s) 1311 for the robotic device 100 to communicate with the display device 200 via network(s), I/O interface(s) 1312 (e.g., a physical button), and the like. The actuation subunit 132 is coupled to component(s)/device(s) for implementing the motions of the robotic device 100 that include the motors M by, for example, actuating the motors M of joints of the robotic device 100. Each of the motors M includes an encoder E for tracking a current position of the motor M. The communication subunit 131 may include controllers for the above-mentioned communication interfaces of the robotic device 100, and the actuation subunit 132 may include controller(s) for the above-mentioned component(s)/device(s) for implementing the motions of the robotic device 100. In other embodiments, the communication subunit 131 and/or actuation subunit 132 may just abstract component for representing the logical relationships between the components of the robotic device 100. In some embodiments, the low-level firmware of the control unit 130 may continuously send the current reading of the encoder E and the force sensor S to the high-level control system (e.g., the extremity rehabilitation module 121).

The robotic device 100 may further include a sensor subunit 133 which may include a set of sensor(s) and related controller(s), for example, the force sensor S, for detecting forces from the user U. The sensor subunit 133 communicates with the control unit 110 over one or more communication buses or signal lines that may be the same or at least partially different from the above-mentioned one or more communication buses or signal lines L. In some embodiments, the various components shown in FIG. 3 may be implemented in hardware, software or a combination of both hardware and software. Two or more of the processing unit 110, the storage unit 120, the control unit 110, the extremity rehabilitation module 121, and other units/subunits/modules may be implemented on a single chip or a circuit. In other embodiments, the sensor subunit 133 may further include a camera and an IMU (inertial measurement unit) (or an accelerometer and a gyroscope), for detecting the situation of the user U to facilitate the above-mentioned upper limb rehabilitation functions. The sensor subunit 133 may just abstract component for representing the logical relationships between the components of the robotic device 100. In addition, at least a part of them may be implemented on separate chips or circuits.

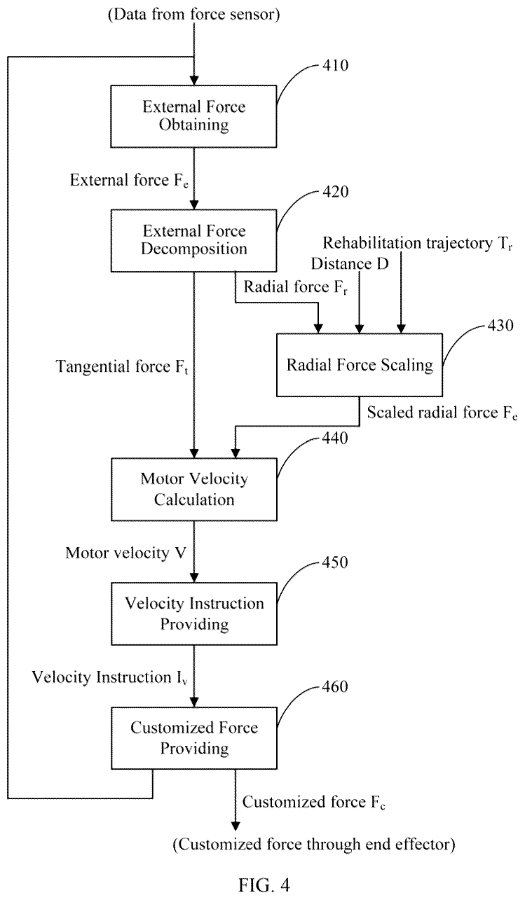

FIG. 4 is a schematic block diagram of an example of performing extremity rehabilitation using the robotic device 100 of FIG. 3. In some embodiments, an upper limb rehabilitation process is implemented in the robotic device 100 to, with the auxiliary of the display device 200, assist the user U to perform the upper limb rehabilitation activities by, for example, storing (sets of) instructions corresponding to the upper limb rehabilitation process (e.g., instructions for controlling the motors M) as the extremity rehabilitation module 121 in the storage unit 120 and executing the stored instructions through the processing unit 110, and then the robotic device 100 may be controlled accordingly. The upper limb rehabilitation process may be performed in response to actuating the robotic device 100 through, for example, physical button(s) or a remote control of the robotic device 100 or the display device 200. In other embodiments, the upper limb rehabilitation process may also be performed in response to a request from, for example, (the operation system of) the robotic device 100 or the display device 200.

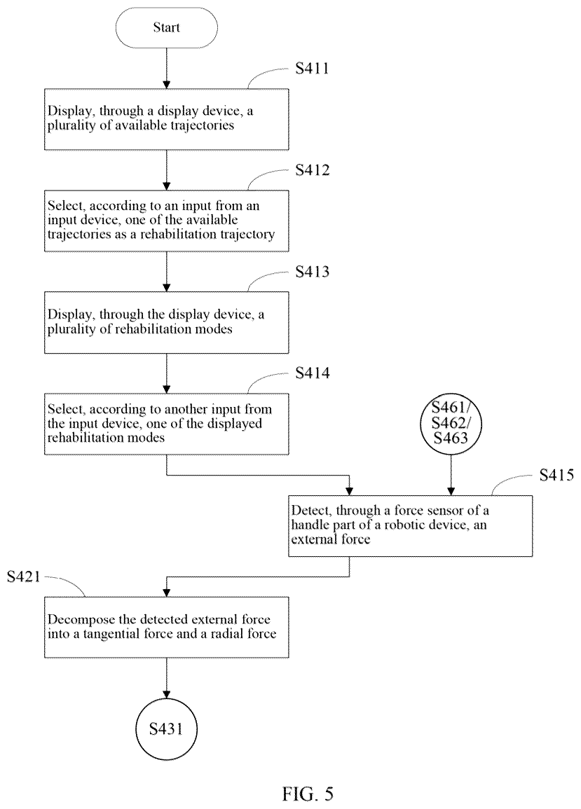

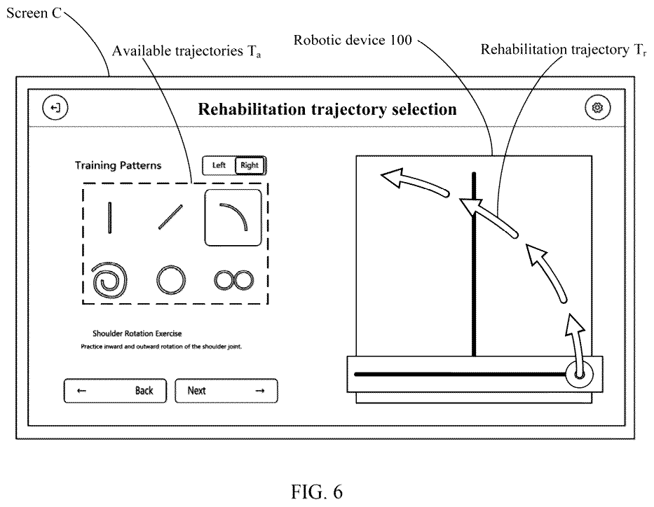

According to the upper limb rehabilitation process, the processing unit 110 may obtain an external force Fe based on (sensor) data received from the force sensor S (block 410 of FIG. 3). FIG. 5 is a flow chart of an example of obtaining and decomposing the external force Fe according to some embodiments of the present disclosure. Accordingly, at step S411, a plurality of available trajectories Tₐ are displayed through the display device 200. FIG. 6 is a schematic diagram of a screen C for selecting a rehabilitation trajectory Tᵣ according to some embodiments of the present disclosure. The screen C is displayed by the display device 200 in response to, for example, the starting of the upper limb rehabilitation process or a request from the user U. Training patterns for upper limbs including a left arm and a right arm may be shown as a plurality of trajectories for shoulder rotation exercise (i.e., the available trajectories Tₐ). The trajectories may be shown as options in textual/graphical manner for the user U to select. As shown in FIG. 6, the left part shows the available trajectories Tₐ to be selected, and the right part shows the selected rehabilitation trajectory Tᵣ on the robotic device 100.

At step S412, one of the available trajectories Tₐ is selected as the rehabilitation trajectory Tᵣ by the user U through, for example, the above-mentioned physical button(s) or remote control of the robotic device 100 or the display device 200 that generates an input indicating the result of selection. At step S413, a plurality of rehabilitation modes Mᵣ (not shown) are displayed through the display device 200. The rehabilitation modes Mᵣ for assisting the user U in performing extremity rehabilitatio may be displayed through the display device 200 as options in textual/graphical manner, so that the user U can select one of the displayed rehabilitation modes Mᵣ as the rehabilitation mode Mᵣ of the robotic device 100 through, for example, the above-mentioned physical button(s) or remote control of the robotic device 100 or the display device 200. The rehabilitation modes Mᵣ may include a free mode representing that the user U controls the robotic device 100 without any assistance or force, an assist mode representing that the robotic device 100 provides assistance or force to the user U, and a resist mode representing that the robotic device 100 applies force to the user U so that the user U needs to add strength to control the robotic device 100. At step S414, one of the displayed rehabilitation modes Mᵣ is selected as the rehabilitation mode Mᵣ of the robotic device 100 by the user U through, for example, the above-mentioned physical button(s) or remote control of the robotic device 100 or the display device 200 that generates an input indicating the result of selection. At step S415, the external force Fₑ is detected through the force sensor S. In some embodiments, the force sensor S may be a two-axis torque sensor that can detect the torque acting on the hand holder 103 at the x-axis direction and the y-axis direction simultaneously. In other embodiments, the force sensor may be other device such as a torque gauge to measure the output torque of the hand holder 103.

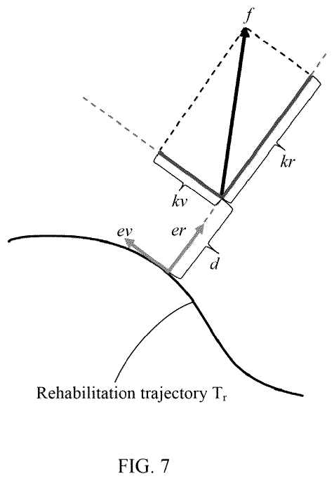

According to the upper limb rehabilitation process, the processing unit 110 may further decompose (i.e., resolute) the detected external force Fe into a tangential force Fₜ and a radial force Fᵣ (block 420 of FIG. 3). FIG. 7 is a schematic diagram of decomposing the external force Fₑ according to some embodiments of the present disclosure. At step S421, the detected external force Fₑ (denoted as f in FIG. 7) may be decomposed into the tangential force Fₜ and the radial force Fᵣ, where the tangential force Fₜ may be represented as an equation of:

$$kv * ev ;$$

where, kv represents a coefficient of the external force Fₑ on a tangential direction at a location of a current trajectory T_{c} (not shown) of the hand holder 103 that corresponds to the current position of the hand holder 103, ev represents a unit direction vector of the external force Fₑ on the tangential direction at the location of the current trajectory of the hand holder 103; and the radial force Fᵣ may be represented as an equation of:

$$kr * er ;$$

where, kr represents a coefficient of the external force Fₑ on a radial direction at the location of the current trajectory of the hand holder 103, and er represents a unit direction vector of the external force Fₑ on the radial direction at the location of the current trajectory of the hand holder 103. The decomposition of the detected external force Fₑ into the tangential force Fₜ and the radial force Fᵣ may be represented as an equation of: (fₓ, f_{y})→kv*ev+kr*er; where fₓ and f_{y} represent the reading of the force sensor S on the x-axis and the y-axis, respectively.

According to the upper limb rehabilitation process, the processing unit 110 may further scale the radial force according to a distance D (denoted as d in FIG. 7) between the current position of the hand holder 103 and the rehabilitation trajectory Tᵣ of the hand holder 103 (block 430 of FIG. 3). At step S431, positional information of the x-axis motor M and that of the y-axis motor M may be obtained from the encoder E of each motor. At step S432, the current position of the hand holder 103 may be determined based on the obtained positional information of each motor. At step S433, the coefficient kr of the radial force Fᵣ may be scaled according to the distance D between the current position of the hand holder 103 and the rehabilitation trajectory Tᵣ of the hand holder 103, where the radial force may be scaled using an equation of:

adaptive_scale(kr, d);

where, d represents the distance D between the current position of the hand holder 103 on the current trajectory T_{c} and the rehabilitation trajectory Tᵣ of the hand holder 103, and adaptive_scale( ) is a scaling function for scaling the radial force Fᵣ so that the larger the distance d, the smaller the scaled radial force Fᵣ.

According to the upper limb rehabilitation process, the processing unit 110 may further calculate a motor velocity V based on a sum of the tangential force Fₜ and the scaled radial force Fᵣ (block 440 of FIG. 3). The motor velocity V is the velocity applied to motor(s) to generate a corresponding force by rotation. At step S441, the motor velocity V may be calculated based on the sum of the tangential force Fₜ and the scaled radial force Fᵣ as an equation of:

$$v = h\_motor ( kv * ev + adaptive\_scale ( kr , d ) ;$$

where, v is the motor velocity V, h_motor( ) is a transfer function from force to motor velocity. Since the end effector (i.e., the base board 101 and the movable frame 102) of the robotic device 100 includes the x-axis motor M and the y-axis motor M that generate forces in the x-axis direction and the y-axis direction, respectively, the calculated motor velocity V may include an x-axis direction velocity and a y-axis direction velocity.

The processing unit 110 may further provide velocity instruction(s) Iᵥ based on the calculated motor velocity V (block 450 of FIG. 3). The velocity instruction(s) Iᵥ may be provided by generating the velocity instruction(s) Iᵥ to transmit to a controller of each motor. At step S451, the velocity instruction Iᵥ may be provided based on the calculated motor velocity V for each of the x-axis motor M and the y-axis motor M. In some embodiments, the velocity instruction Iᵥ for the x-axis motor M may be provided based on the x-axis direction velocity, and the velocity instruction Iᵥ for the y-axis motor M may be provided based on the y-axis direction velocity.

The processing unit 110 may further provide a customized force F_{c} corresponding to the detected external force Fₑ through moving the end effector (i.e., the base board 101 and the movable frame 102) of the robotic device 100 by controlling the motor(s) of the end effector to rotate according to the provided velocity instruction(s) Iᵥ (block 460 of FIG. 3). At step S461, it determines the rehabilitation mode Mᵣ of the robotic device 10. If the rehabilitation mode Mᵣ is the free mode, it directly backs to step S415; if the rehabilitation mode Mᵣ is the assist mode, step S462 will be performed; and if the rehabilitation mode Mᵣ is the resist mode, step S463 will be performed. At step S462, the customized force F_{c} corresponding to the external force Fₑ is provided by controlling each of the x-axis motor M and the y-axis motor M to rotate forwardly according to the provided velocity instructions Iᵥ. The forward rotation means rotating in the direction to provide a force with the same direction as the external force Fₑ as the customized force F_{c}. At step S463, the customized force Fₑ corresponding to the external force Fₑ is provided by controlling each of the x-axis motor M and the y-axis motor M to rotate reversely according to the provided velocity instructions Iᵥ. The reverse rotation means rotating in the direction to provide a counter force of the external force Fₑ as the customized force Fₑ. The customized force Fₑ corresponding to the external force Fₑ may be provided by controlling the x-axis motor M to rotate according to the velocity instruction Iᵥ for the x-axis motor M and controlling the y-axis motor M to rotate according to the velocity instruction Iᵥ for the y-axis motor M. After performing the customized force providing steps, it backs to step S415 for continuing the detection of the external force Fₑ.

The robotic device 100 is capable of dynamically adapt force assistance or resistance according to the difference between the actual trajectory (i.e., the current trajectory T_{c}) of the user's extremity with the desired rehabilitation trajectory (i.e., the selected rehabilitation trajectory Tᵣ). In comparison with the existing extremity rehabilitation devices, by using a single sensor which is common in the related technology, that is, the force sensor S, the control of the robotic device 100 for assisting the user U in performing the upper limb rehabilitation activities can be realized in a simpler and lower-cost manner.

It can be understood by those skilled in the art that, all or part of the method in the above-mentioned embodiment(s) can be implemented by one or more computer programs to instruct related hardware. In addition, the one or more programs can be stored in a non-transitory computer readable storage medium. When the one or more programs are executed, all or part of the corresponding method in the above-mentioned embodiment(s) is performed. Any reference to a storage, a memory, a database or other medium may include non-transitory and/or transitory memory. Non-transitory memory may include read only memory (ROM), programmable ROM (PROM), electrically programmable ROM (EPROM), electrically erasable programmable ROM (EEPROM), flash memory, solid-state drive (SSD), or the like. Volatile memory may include random access memory (RAM), external cache memory, or the like.

The processing unit 110 (and the above-mentioned processor) may include central processing unit (CPU), or be other general purpose processor, graphics processing unit (GPU), digital signal processor (DSP), application specific integrated circuit (ASIC), field-programmable gate array (FPGA), or be other programmable logic device, discrete gate, transistor logic device, and discrete hardware component. The general purpose processor may be microprocessor, or the processor may also be any conventional processor. The storage unit 120 (and the above-mentioned memory) may include internal storage unit such as hard disk and internal memory. The storage unit 120 may also include external storage device such as plug-in hard disk, smart media card (SMC), secure digital (SD) card, and flash card.

The exemplificative units/modules and methods/steps described in the embodiments may be implemented through software, hardware, or a combination of software and hardware. Whether these functions are implemented through software or hardware depends on the specific application and design constraints of the technical schemes. The above-mentioned path planning method and mobile machine may be implemented in other manners. For example, the division of units/modules is merely a logical functional division, and other division manner may be used in actual implementations, that is, multiple units/modules may be combined or be integrated into another system, or some of the features may be ignored or not performed. In addition, the above-mentioned mutual coupling/connection may be direct coupling/connection or communication connection, and may also be indirect coupling/connection or communication connection through some interfaces/devices, and may also be electrical, mechanical or in other forms.

The above-mentioned embodiments are merely intended for describing but not for limiting the technical schemes of the present disclosure. Although the present disclosure is described in detail with reference to the above-mentioned embodiments, the technical schemes in each of the above-mentioned embodiments may still be modified, or some of the technical features may be equivalently replaced, so that these modifications or replacements do not make the essence of the corresponding technical schemes depart from the spirit and scope of the technical schemes of each of the embodiments of the present disclosure, and should be included within the scope of the present disclosure.

What is claimed is:

1. A method for a robotic device having an end effector including one or more motors and a handle part including a force sensor, wherein the handle part is coupled to the end effector for moving the end effector; the method comprises:
detecting, through the force sensor of the handle part of the robotic device that is coupled to the end effector of the robotic device for moving the end effector, an external force;
decomposing the detected external force into a tangential force and a radial force;
scaling, according to a distance between a current position of the handle part and a desired trajectory of the handle part, the radial force;
calculating, based on a sum of the tangential force and the scaled radial force, a motor velocity for each of the one or more motors; and
providing a velocity instruction based on the calculated motor velocity for rotating the one or more motors of the end effector of the robotic device to move the end effector.

2. The method of claim 1, wherein the tangential force is represented as an equation of:
$$kv * ev ;$$
where, kv represents a coefficient of the external force on a tangential direction at a location of a current trajectory of the handle part that corresponds to the current position of the handle part, ev represents a unit direction vector of the external force on the tangential direction at the location of the current trajectory of the handle part; and
the radial force is represented as an equation of:
$$kr * er ;$$
where, kr represents a coefficient of the external force on a radial direction at the location of the current trajectory of the handle part, and er represents a unit direction vector of the external force on the radial direction at the location of the current trajectory of the handle part.

3. The method of claim 2, wherein the radial force is scaled using an equation of:
adaptive_scale(kr, d);
where, d represents the distance between the current position of the handle part and the desired trajectory of the handle part, and adaptive_scale( ) is a scaling function for scaling the radial force so that the larger the distance d, the smaller the scaled radial force.

4. The method of claim 3, wherein the motor velocity is calculated using an equation of:
$$v = h\_motor ( kv * ev + adaptive\_scale ( kr , d ) ;$$
where, v is the motor velocity, h_motor( ) is a transfer function from force to motor velocity.

5. The method of claim 1, further comprising:
providing a customized force corresponding to the detected external force through moving the end effector of the robotic device by controlling the one or more motors of the end effector to rotate according to the provided velocity instruction.

6. The method of claim 5, wherein the end effector is composed of a first end effector part and a second end effector part slidably disposed on the first end effector part; the handle part is slidably disposed on the second end effector part, and is selectively slid in an x-direction; the second end effector part is selectively slid in a y-direction perpendicular to the x-direction; and the first end effector part includes a first motor for being rotated to move the second end effector part along the y-direction, and the second end effector part includes a second motor for being rotated to move the handle part along the x-direction; wherein providing the customized force corresponding to the detected external force through moving the end effector of the robotic device by controlling the one or more motors of the end effector to rotate according to the provided velocity instruction comprises:
providing the customized force corresponding to the detected external force through at least one of moving the handle part along the x-direction by controlling the second motor of the second end effector part to rotate according to the provided velocity instruction and moving the second end effector part along the y-direction by controlling the first motor of the first end effector part to rotate according to the provided velocity instruction.

7. The method of claim 5, wherein the robotic device is an extremity rehabilitation device for assisting a user in performing extremity rehabilitation by using an assistance force as the customized force in an assist mode and a resistance force as the customized force in a resist mode, wherein providing the customized force corresponding to the detected external force through moving the end effector of the robotic device by controlling the one or more motors of the end effector to rotate according to the provided velocity instruction comprises:
providing the assistance force proportional to the detected external force through moving the end effector of the robotic device by controlling the one or more motors of the end effector to rotate forwardly according to the provided velocity instruction, in response to the extremity rehabilitation device being in the assist mode; and
providing the resistance force inversely proportional to the detected external force through moving the end effector of the robotic device by controlling the one or more motors of the end effector to rotate reversely according to the provided velocity instruction, in response to the extremity rehabilitation device being in the resist mode.

8. The method of claim 1, wherein each of the one or more motors includes an encoder, and the current position of the handle part is determined by:
obtaining, from the encoder of at least one of the one or more motors, positional information of the motor; and
determining, based on the obtained positional information of at least one of the one or more motors, the current position of the handle part.

9. The method of claim 1, further comprising:
obtaining, according to an input from an input device communicatively connected with the robotic device, the desired trajectory of the handle part.

10. The method of claim 9, further comprising:
displaying, through a display device communicatively connected with the robotic device, a plurality of available trajectories;
obtaining, according to the input from the input device communicatively connected with the robotic device, the desired trajectory of the handle part comprises:
selecting, according to the input from the input device communicatively connected with the robotic device, one of the available trajectories as the desired trajectory.

11. A robotic device, comprising:
a first end effector part including a first motor;
a second end effector part slidably disposed on the first end effector part, wherein the second end effector part includes a second motor, and is selectively slid in a y-direction; wherein the first motor of the first end effector part is for being rotated to move the second end effector part along the y-direction;
a handle part slidably disposed on the second end effector part, wherein the handle part includes a force sensor, and is selectively slid in an x-direction perpendicular to the y-direction; wherein the second motor of the second end effector part is for being rotated to move the handle part along the x-direction;
one or more processors; and
one or more memories storing one or more programs configured to be executed by the one or more processors, wherein the one or more programs comprise instructions to:
detect, through the force sensor of the handle part, an external force;
decompose the detected external force into a tangential force and a radial force;
scale, according to a distance between a current position of the handle part and a desired trajectory of the handle part, the radial force;
calculate, based on a sum of the tangential force and the scaled radial force, a motor velocity for each of the first motor and the second motor;
provide a velocity instruction based on the calculated motor velocity for each of the first motor and the second motor; and
provide a customized force corresponding to the detected external force through at least one of moving the handle part along the x-direction by controlling the second motor of the second end effector part to rotate according to the provided velocity instruction and moving the second end effector part along the y-direction by controlling the first motor of the first end effector part to rotate according to the provided velocity instruction.

12. The robotic device of claim 11, wherein the tangential force is represented as an equation of:
$$kv * ev ;$$
where, kv represents a coefficient of the external force on a tangential direction at a location of a current trajectory of the handle part that corresponds to the current position of the handle part, ev represents a unit direction vector of the external force on the tangential direction at the location of the current trajectory of the handle part; and
the radial force is represented as an equation of:
$$kr * er ;$$
where, kr represents a coefficient of the external force on a radial direction at the location of the current trajectory of the handle part, and er represents a unit direction vector of the external force on the radial direction at the location of the current trajectory of the handle part.

13. The robotic device of claim 12, wherein the radial force is scaled using an equation of:
adaptive_scale(kr, d);
where, d represents the distance between the current position of the handle part and the desired trajectory of the handle part, and adaptive_scale( ) is a scaling function for scaling the radial force so that the larger the distance d, the smaller the scaled radial force.

14. The robotic device of claim 13, wherein the motor velocity is calculated using an equation of:
$$v = h\_motor ( kv * ev + adaptive\_scale ( kr , d ) ;$$
where, v is the motor velocity, h_motor( ) is a transfer function from force to motor velocity.

15. The robotic device of claim 11, wherein the one or more programs further comprise instructions to:
provide the customized force corresponding to the detected external force through at least one of moving the handle part along the x-direction by controlling the second motor of the second end effector part to rotate according to the provided velocity instruction and moving the second end effector part along the y-direction by controlling the first motor of the first end effector part to rotate according to the provided velocity instruction.

16. The robotic device of claim 15, wherein the robotic device is an extremity rehabilitation device for assisting a user in performing extremity rehabilitation by using an assistance force as the customized force in an assist mode and a resistance force as the customized force in a resist mode, wherein providing the customized force corresponding to the detected external force through at least one of moving the handle part along the x-direction by controlling the second motor of the second end effector part to rotate according to the provided velocity instruction and moving the second end effector part along the y-direction by controlling the first motor of the first end effector part to rotate according to the provided velocity instruction comprises:
providing the assistance force proportional to the detected external force through at least one of moving the handle part along the x-direction by controlling the second motor of the second end effector part to rotate forwardly according to the provided velocity instruction and moving the second end effector part along the y-direction by controlling the first motor of the first end effector part to rotate forwardly according to the provided velocity instruction, in response to the extremity rehabilitation device being in the assist mode; and
providing the resistance force inversely proportional to the detected external force through at least one of moving the handle part along the x-direction by controlling the second motor of the second end effector part to rotate reversely according to the provided velocity instruction and moving the second end effector part along the y-direction by controlling the first motor of the first end effector part to rotate reversely according to the provided velocity instruction, in response to the extremity rehabilitation device being in the resist mode.

17. The robotic device of claim 11, wherein each of the one or more motors includes an encoder, and the current position of the handle part is determined by:
obtaining, from the encoder of at least one of the one or more motors, positional information of the motor; and
determining, based on the obtained positional information of at least one of the one or more motors, the current position of the handle part.

18. The robotic device of claim 11, the one or more programs further comprise instructions to:
obtain, according to an input from an input device communicatively connected with the robotic device, the desired trajectory of the handle part.

19. The robotic device of claim 18, the one or more programs further comprise instructions to:
display, through a display device communicatively connected with the robotic device, a plurality of available trajectories;
obtaining, according to the input from the input device communicatively connected with the robotic device, the desired trajectory of the handle part comprises:
selecting, according to the input from the input device communicatively connected with the robotic device, one of the available trajectories as the desired trajectory.
